(19) United States
(12) Patent Application Publication
(10) Pub. No.: US 2021/0348062 A1
(43) Pub. Date: Nov. 11, 2021

(54) CONVERSION OF SUGARS TO HYDROCARBONS VIA A FATTY ALCOHOL INTERMEDIATE

(71) Applicant: REG SYNTHETIC FUELS, LLC, Ames, IA (US)

(72) Inventors: David A. Slade, Ames, IA (US); Matt Herman, Ames, IA (US); Martin Haverly, Ames, IA (US); Ramin Abhari, Bixby, OK (US)

(21) Appl. No.: 17/286,130

(22) PCT Filed: Oct. 16, 2019
(86) PCT No.: PCT/US2019/056458

§ 371 Date: Apr. 16, 2021

Related U.S. Application Data
(60) Provisional application No. 62/748,216, filed on Oct. 19, 2018.

(51) Int. Cl. C10G 3/00; B01J 23/30; C12P 7/04; C10G 49/06; B01J 23/42; C10G 65/02

(52) U.S. Cl. CPC C10G 3/44; B01J 23/30; C10G 3/50; C12P 7/04; C10G 49/06; B01J 37/20; C10G 65/02; C10G 2300/1011; C10G 2300/4006; C10G 2300/4012; B01J 23/42

(57) ABSTRACT

The present technology provides a method to produce hydrocarbon renewable fuels. The method includes hydrodeoxygenating a feed to produce a hydrocarbon product, where the feed includes fatty alcohols and the hydrocarbon product includes C₁₀-C₁₂ n-paraffins and a heteroatom oxygen content less than 0.1 wt %.

### CROSS-REFERENCE TO RELATED APPLICATION

This application claims the benefit of and priority to U.S. Provisional Application No. 62/748,216, filed Oct. 19, 2018, the entirety of which is incorporated herein by reference for any and all purposes.

### SUMMARY

In an aspect, the present technology provides a method to produce hydrocarbon renewable fuels. The method includes hydrodeoxygenating a feed to produce a hydrocarbon product, where the feed includes fatty alcohols and the hydrocarbon product includes C₁₀-C₁₂ n-paraffins and a heteroatom oxygen content less than 0.1 wt %.

### BRIEF DESCRIPTION OF THE DRAWINGS

FIG. 1 illustrates a non-limiting illustration of a method of the present technology.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt %” means “9 wt % to 11 wt %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “alkyl” groups include straight chain and branched alkyl groups. Examples of straight chain alkyl groups include methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, neopentyl, and isopentyl groups. It will be understood that the phrase “Cₓ-C_{y} alkyl,” such as C₁-C₄ alkyl, means an alkyl group with a carbon number falling in the range from x to y.

The term “aromatics” as used herein is synonymous with “aromates” and means both cyclic aromatic hydrocarbons that do not contain heteroatoms as well as heterocyclic aromatic compounds. The term includes monocyclic, bicyclic and polycyclic ring systems (collectively, such bicyclic and polycyclic ring systems are referred to herein as “polycyclic aromatics” or “polycyclic aromates”). The term also includes aromatic species with alkyl groups and cycloalkyl groups. Thus, aromatics include, but are not limited to, benzene, azulene, heptalene, phenylbenzene, indacene, fluorene, phenanthrene, triphenylene, pyrene, naphthacene, chrysene, anthracene, indene, indane, pentalene, and naphthalene, as well as alkyl and cycloalkyl substituted variants of these compounds. In some embodiments, aromatic species contains 6-14 carbons, and in others from 6 to 12 or even 6-10 carbon atoms in the ring portions of the groups. The phrase includes groups containing fused rings, such as fused aromatic-aliphatic ring systems (e.g., indane, tetrahydronaphthene, and the like).

“Oxygenates” as used herein means carbon-containing compounds containing at least one covalent bond to oxygen. Examples of functional groups encompassed by the term include, but are not limited to, carboxylic acids, carboxylates, acid anhydrides, aldehydes, esters, ethers, ketones, and alcohols, as well as heteroatom esters and anhydrides such as phosphate esters and phosphate anhydrides. Oxygenates may also be oxygen containing variants of aromatics, cycloparaffins, and paraffins as described herein.

The term “paraffins” as used herein means non-cyclic, branched or unbranched alkanes. An unbranched paraffin is an n-paraffin; a branched paraffin is an iso-paraffin. “Cycloparaffins” are cyclic, branched or unbranched alkanes.

The term “paraffinic” as used herein means both paraffins and cycloparaffins as defined above as well as predominantly hydrocarbon chains possessing regions that are alkane, either branched or unbranched, with mono- or di-unsaturation (i.e., one or two double bonds).

Hydroprocessing as used herein describes the various types of catalytic reactions that occur in the presence of hydrogen without limitation. Examples of the most common hydroprocessing reactions include, but are not limited to, hydrogenation, hydrodesulfurization (HDS), hydrodenitrogenation (HDN), hydrotreating (HT), hydrocracking (HC), aromatic saturation or hydrodearomatization (HDA), hydrodeoxygenation (HDO), decarboxylation (DCO), hydroisomerization (HI), hydrodewaxing (HDW), hydrodemetallization (HDM), decarbonylation, methanation, and reforming. Depending upon the type of catalyst, reactor configuration, reactor conditions, and feedstock composition, multiple reactions can take place that range from purely thermal (i.e., do not require catalyst) to catalytic. In the case of describing the main function of a particular hydroprocessing unit, for example an HDO reaction system, it is understood that the HDO reaction is merely one of the predominant reactions that are taking place and that other reactions may also take place.

Decarboxylation (DCO) is understood to mean hydroprocessing of an organic molecule such that a carboxyl group is removed from the organic molecule to produce CO₂, as well as decarbonylation which results in the formation of CO.

Pyrolysis is understood to mean thermochemical decomposition of carbonaceous material with little to no diatomic oxygen or diatomic hydrogen present during the thermochemical reaction. The optional use of a catalyst in pyrolysis is typically referred to as catalytic cracking, which is encompassed by the term as pyrolysis, and is not be confused with hydrocracking.

Hydrotreating (HT) involves the removal of elements from groups 3, 5, 6, and/or 7 of the Periodic Table from organic compounds. Hydrotreating may also include hydrodemetallization (HDM) reactions. Hydrotreating thus involves removal of heteroatoms such as oxygen, nitrogen, sulfur, and combinations of any two more thereof through hydroprocessing. For example, hydrodeoxygenation (HDO) is understood to mean removal of oxygen by a catalytic hydroprocessing reaction to produce water as a by-product; similarly, hydrodesulfurization (HDS) and hydrodenitrogenation (HDN) describe the respective removal of the indicated elements through hydroprocessing.

Hydrogenation involves the addition of hydrogen to an organic molecule without breaking the molecule into subunits. Addition of hydrogen to a carbon-carbon or carbon-oxygen double bond to produce single bonds are two nonlimiting examples of hydrogenation. Partial hydrogenation and selective hydrogenation are terms used to refer to hydrogenation reactions that result in partial saturation of an unsaturated feedstock. For example, vegetable oils with a high percentage of polyunsaturated fatty acids (e.g., linoleic acid) may undergo partial hydrogenation to provide a hydroprocessed product wherein the polyunsaturated fatty acids are converted to mono-unsaturated fatty acids (e.g., oleic acid) without increasing the percentage of undesired saturated fatty acids (e.g., stearic acid). While hydrogenation is distinct from hydrotreatment, hydroisomerization, and hydrocracking, hydrogenation may occur amidst these other reactions.

Hydrocracking (HC) is understood to mean the breaking of a molecule's carbon-carbon bond to form at least two molecules in the presence of hydrogen. Such reactions typically undergo subsequent hydrogenation of the resulting double bond.

Hydroisomerization (HI) is defined as the skeletal rearrangement of carbon-carbon bonds in the presence of hydrogen to form an isomer. Hydrocracking is a competing reaction for most HI catalytic reactions and it is understood that the HC reaction pathway, as a minor reaction, is included in the use of the term HI. Hydrodewaxing (HDW) is a specific form of hydrocracking and hydroisomerization designed to improve the low temperature characteristics of a hydrocarbon fluid.

It will be understood that if a composition is stated to include “Cₓ-C_{y} hydrocarbons,” such as C₇-C₁₂ n-paraffins, this means the composition includes one or more paraffins with a carbon number falling in the range from x to y.

A “diesel fuel” in general refers to a fuel with boiling point that falls in the range from about 150° C. to about 360° C. (the “diesel boiling range”).

A “biodiesel” as used herein refers to fatty acid C₁-C₄ alkyl esters produced by esterification and/or transesterification reactions between a C₁-C₄ alkyl alcohol and free fatty acids and/or fatty acid glycerides, such as described in U.S. Pat. Publ. No. 2016/0145536, incorporated herein by reference.

A “petroleum diesel” as used herein refers to diesel fuel produced from crude oil, such as in a crude oil refining facility and includes hydrotreated straight-run diesel, hydrotreated fluidized catalytic cracker light cycle oil, hydrotreated coker light gasoil, hydrocracked FCC heavy cycle oil, and combinations thereof.

It is to be understood that a “volume percent” or “vol. %” of a component in a composition or a volume ratio of different components in a composition is determined at 60° F. based on the initial volume of each individual component, not the final volume of combined components.

Renewable diesel (RD) is an paraffinic compression ignition fuel produced by hydroprocessing. The process typically includes hydrodeoxygenation of fats and oils to hydrocarbons rich in n-paraffins followed by hydroisomerization. Commercial production of RD began in 2008 and has grown to about 1.5 billion gal/y worldwide in just ten years. The growth of RD production capacity is expected to continue and disrupt lipid supply-demand balance. As such, there is a need to explore use of non-conventional lipid feedstocks for RD production.

The present technology is based, in part, on the surprising discovery that the oil phase from microbial fermentation of sugars is an advantageous renewable feed for production of RD. Such microbial fermentation process have been described in, e.g., U.S. Pat. No. 9,598,706. For example, the oil phase includes C₁₂ and C₁₄ fatty alcohols (“FALC”) and, upon hydrodeoxygenation and subsequent hydroisomerization of this oil phase, provides a higher hydrocarbon yield and lower H₂ consumption. Eq 1 illustrates hydrodeoxygenation of oleic acid (the major component in conventional fats/oil where it exists as a glyceride or a free fatty acid), whereas Eq 2 shows tetradecanol (a component of the feed of the present technology).

C₁₇H₃₃—COOH (oleic acid)+4H₂→C₁₈H₃₈ (octadecane)+2H₂O (1)

Stoichiometric hydrocarbon yield=254.5/282.5=90.1%

H₂ consumption=4 mol H₂/254.5 g hydrocarbon product=15.7 gmol/kg

C₁₄H₂₉—OH (tetradecanol)+H₂→C₁₄H₃₀ (tertadecane)+H₂O (2)

Stoichiometric hydrocarbon yield=198.5/214.3=92.6%

H₂ consumption=1 mol H₂/198.5 g hydrocarbon product=5.04 gmol/kg

The fatty acid HDO reaction of Eq 1 may be accompanied by decarboxylation (Eq 3) and decarbonylation (Eq 4) side reactions whereby oxygen is removed as CO and CO₂ instead of water.

C₁₇H₃₃—COOH (oleic acid)+H₂→C₁₈H₃₆ (heptadecane)+CO₂ (3)

C₁₇H₃₃—COOH (oleic acid)+2H₂→C₁₈H₃₆ (heptadecane)+CO+H₂O (4)

The disadvantage of these “decarb” reactions is reduced yield (loss of carbon atom from the fatty acid chain) and need to remove CO/CO₂ from recycle hydrogen. The FALC HDO reaction of Eq 2 does not have a corresponding “decarb” reaction and thus provides additional yield and processing advantages.

Furthermore, feedstocks comprising FALC yield a hydrodeoxygenated product that meets the diesel cloud point requirements of many regions, typically eliminating the need for hydroisomerization. Given the potential use of cellulosic feeds as source of sugars for the fermentation step, and the lower HDO hydrogen consumption associated with the fermentation product comprising fatty alcohols, the present invention provides a lower carbon intensity pathway to both fatty alcohols and renewable diesel compared to prior art methods. Carbon intensity is a measure of life-cycle greenhouse gas emissions. For renewable fuel from the present technology, the carbon intensity is between about 60% and about 90% lower than ultralow sulfur diesel refined from petroleum.

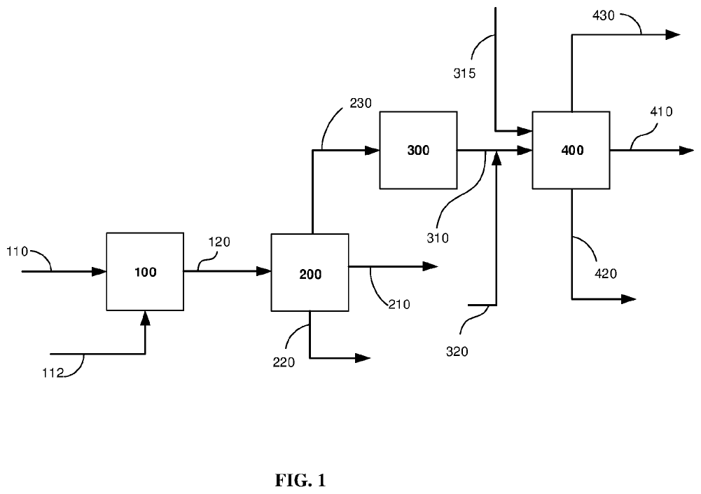

FIG. 1 provides a non-limiting illustration of a method of the present technology. Referring to FIG. 1, a sugar feedstock 110 is fermented in fermenter 100 where it is contacted with oxygen from air. The sugar feedstock 110 may include, but is not limited to, a C₅ sugar, a C₆ sugar, an anhydrosugar, a polysaccharide including any one or more of the aforementioned, a hydrolyzed product of a any one or more of the aforementioned, a pyrolysis product of any one or more of the aforementioned, or a combination of any two or more thereof. More particular examples include, but are not limited to, glucose (e.g., extracted from corn, sugar beets, sugar cane, palm sugar, or a combination of any two or more thereof), sugars and/or anhydrosugars recovered from the cellulosic portion of biomass (such as stalks, branches, tree trunks, or a combination of any two or more thereof), sugars and/or anhydrosugars recovered from hydrolysis of biomass (such as cellulose, hemi-cellulose, or a combination thereof). Anhydrosugars may also be provided from thermal decomposition of biomass, and may or may not subsequently be hydrolyzed to produce simple sugars. Depending on enzyme or bacteria used to catalyze the fermentation in fermenter 100, the sugar will undergo different bio-synthetic pathways. Bacteria (e.g., E. coli strains) may promote production of fatty acid derivatives including esters and alcohols, as described in U.S. Pat. No. 9,598,706. The bio-synthetic conversion in fermenter 100 may occur at a temperature of about 90° F. to about 110° F. in water with the addition of oxygen (such as air injection stream 112) and evolution of CO₂ as the bacteria synthesize and secrete fatty alcohols and optionally as well as one or more of free fatty acids, lipids, triglycerides, etc. The fermenter may further be agitated to promote diffusion of oxygen to the bacteria and suspension of oil phase droplets (including fatty acid esters, fatty alcohols, or a combination thereof) during the fermentation batch cycle. The fermentation batch cycle may vary between a few hours and a few days (typically 12 to 72 hours) and is typically deemed complete when the sugar concentration drops below 1 g/L.

At the end of the batch cycle, a fermentation broth 120 is discharged from the fermenter 100 and washed with water (“wash water”) in oil recovery unit 200 before separation of spent water 210, solid biomass 220 (including microbial fermentation residues), and a recovered FALC-rich oil 230. The oil recovery unit 200 may include a three-phase-centrifuge (e.g., a disc stack centrifuge) where water 210, residual solid biomass 220, and washed FALC-rich oil 230 are separated in one step. Wash water may or may not be added directly to the centrifuge.

FALC-rich oil 230 may include at least 50 wt % of combination of 1-dodecanol and 1-tetradecanol. In any embodiment herein, the 1-dodecanol and 1-tetradecanol may make up from 50 wt % to about 90 wt % of the FALC-rich oil 230. In any embodiment herein, FALC-rich oil 230 may further include at least 2 wt % of a C₁₂ fatty alcohol having one carbon-carbon double bond and at least 1 wt % of a C₁₄ fatty alcohol having one carbon-carbon double bond. In any embodiment herein, FALC-rich oil 230 may include from about 20 wt % to about 50 wt % 1-dodecanol, about 10 wt % to about 40 wt % 1-tetradecanol, about 2 wt % to about 5 wt % of a C₁₂ fatty alcohol having one carbon-carbon double bond, and about 1 wt % to about 3 wt % of a C₁₄ fatty alcohol having one carbon-carbon double bond. In any embodiment herein, FALC-rich oil 230 may further include about 0.1 wt % to about 10 wt % C₁₂-C₁₈ free fatty acids (FFA), such as about 0.1 wt % to about 6 wt % C₁₂-C₁₈ FFA, or such as about 0.1 wt % to about 2 wt % C₁₂-C₁₈ FFA. In any embodiment herein, FALC-rich oil 230 may further include about 1 wt % to about 4 wt % 1-decanol. In any embodiment herein, FALC-rich oil 230 may further include about 0.1 wt % to about 1 wt % C₅-C₁₄ diols. In any embodiment herein, a weight ratio of 1-dodecanol to 1-tetradecanol in the FALC-rich oil may be about 1.2:1 to about 2:1.

In any embodiment herein, the FALC-rich oil 230 may optionally be pretreated in a pretreatment step 300 before being subjected to hydrodeoxygenation (HDO) in HDO reactor system 400 in order to reduce and/or remove contaminants (such as phosphorus and metals) present in the FALC-rich oil and provide a pretreated FALC-rich oil 310 having a phosphorus content of about 10 ppm or less and a total metals content of about 10 ppm or less. Such a pretreatment step may include contacting the FALC-rich oil with an aqueous acid solution, such as citric acid and/or phosphoric acid, and separating insolubles (such as solids and gums) and water through a disc-stack centrifuge system (see, e.g., U.S. Pat. No. 9,783,763). In any embodiment herein, the pretreatment step may include contacting the FALC-rich oil with a filter media powder such as amorphous silica, bleaching clays, ion exchange resins, diatomaceous earth (D.E.) powder, or a combination of any two or more thereof, in a slurry tank and subsequently separating the filter media powder from the (now cleaned) FALC-rich oil in a filter. In embodiments, a blend of amorphous silica and DE are used as the primary filter media. In any embodiment herein, contacting the FALC-rich oil with a filter media powder (e.g., silica/D.E.) may be performed at a temperature of about 150° F. to about 200° F. (such as about 160° F. to about 190° F.) to ensure proper fluid viscosity. In any embodiment herein, contacting the FALC-rich oil with a filter media powder (e.g., silica/D.E.) may be performed at less than about 400 mbar vacuum pressure to ensure proper dehydration of the slurry. In any embodiment herein, the filter media powder (e.g., amorphous silica) may be introduced to the slurry tank at a rate of about 0.1 to about 0.5% (w/w FALC-rich oil flow basis), preferably about 0.3 to about 0.4%. In any embodiment herein, the residence time of FALC-rich oil in the slurry tank may be about 10 minutes to about 90 minutes (such as about 20 minutes to about 50 minutes). Alternatively, in any embodiment herein, it may be that no pretreatment step is performed because FALC-rich oil 230 has less than about 10 ppm phosphorus and about 10 ppm or less total metals.

FALC-rich oil 230, pretreated FALC-rich oil 310, or a combination thereof may be directed to HDO reactor system 400 where it is combined with hydrogen 315 and contacted with a HDO catalyst under hydrogen pressure at a temperature from about 500° F. to about 700° F. to produce hydrocarbon product 410, water effluent 420, and bleed gas 430. Exemplary catalysts and pressures have been described in U.S. Pat. Nos. 7,232,935, 7,968,757, and 8,628,308, where HDO catalysts typically include sulfided supported base metal catalysts, such as Mo, NiMo, and CoMo catalysts, and typically include a H₂ partial pressure of about 500 psig to about 4,000 psig (such as about 1,000 psig to about 2,000 psig H₂ partial pressure). In any embodiment herein, FALC-rich oil 230, pretreated FALC-rich oil 310, or a combination thereof may optionally be combined with HDO co-processing feed 320. The HDO co-processing feed 320 may include a lipid component (such as fats, oil, and/or greases that include fatty acid glycerides and free fatty acids), a biobased crude oil (such as pyrolysis bio-oil from lignocellulosic and/or lipid feedstocks) a petroleum fraction (such a petroleum diesel, a petroleum gas oil, or a combination thereof). When the HDO co-processing feed does not include a petroleum fraction, an organosulfur compound such as dimethyl disulfide may be introduced to streams 230, 310, 320, or a combination of any two or more thereof, to ensure the HDO catalyst is maintained in an active sulfide form.

Hydrocarbon product 410 includes C₁₀-C₁₈ paraffins, such as C₁₀-C₁₄ paraffins, and has a residual elemental oxygen (as heteroatom) of 0.1 wt % of less as measured by fast neutron activation analysis or similar neutron activation methods.

The water effluent (420) includes water made by the HDO reactions of Eq 1, 2 and any other water that was injected in the HDO reactor system for processing purposes as recognized by those skilled in the art (e.g. to wash mineral deposits that can form in recycle hydrogen system). The bleed gas (430) includes any unreacted hydrogen as well as gas phase byproducts (such as ammonia, hydrogen sulfide, carbon dioxide, and carbon monoxide) and may also include C₁-C₄ hydrocarbons.

Depending on the concentration of wax-forming C₁₇₊ n-paraffins in the hydrocarbon product 410, the hydrocarbon product may meet diesel fuel's seasonal/regional cloud point requirements and be used as a compression ignition fuel in neat or blended form. When the C₁₇₊ n-paraffin concentration is higher than 10 wt % or the cloud point is greater than 0° C., additional processing steps may be included in the method to decrease the C₁₇₊ n-paraffin content and reduce the cloud point below 0° C., preferably to about −10° C. or less. A preferred additional processing step is hydroisomerization wherein the long chain n-paraffins are converted to branched isoparaffins as described in, e.g., U.S. Pat. No. 7,968,757, to provide a hydroisomerization product that includes isoparaffins as well as any unreacted n-paraffins. Hydroisomerization is generally conducted in fixed-bed reactors over bifunctional noble metal catalysts (such as platinum) and/or base metal catalysts (such as tungsten) and an acid-support (such as a zeolite). Hydroisomerization may be performed at a temperature of about 580° F. to about 680° F. and may be performed at H₂ partial pressures of about 500 psig to about 2000 psig.

Hydroisomerization is typically accompanied by hydrocracking side reactions, and therefore, in any embodiment herein, the hydroisomerization product may be fractionated to separate the lighter hydrocarbons (naphtha/LPG) that are formed during hydrocracking, such as described in, e.g., U.S. Pat. No. 8,558,042, to provide a hydroisomerizate. The hydroisomerizate may exhibit a cloud point of about −10° C. or less, such as a cloud point of about −10° C. and −30° C.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A method to produce hydrocarbon renewable fuels, the method comprising
   hydrodeoxygenating a feed comprising fatty alcohols to produce a hydrocarbon product comprising C₁₀-C₁₂ n-paraffins and a heteroatom oxygen content less than 0.1 wt %.
B. The method of Paragraph A, wherein the hydrodeoxygenating comprises contacting the feed with a sulfided base metal catalyst at a temperature from about 500° F. to about 700° F. range and a hydrogen partial pressure from about 500 psig to about 4000 psig.
C. The method of Paragraph A or Paragraph B, wherein the hydrocarbon renewable fuel comprises a renewable diesel.
D. The method of any one of Paragraphs A-C, wherein the hydrocarbon renewable fuel comprises a renewable kerosene.
E. The method of any one of Paragraphs A-D, wherein the hydrocarbon renewable fuel comprises a renewable naphtha.
F. The method of any one of Paragraphs A-E, wherein the feed comprises a product of fermentation of sugars.
G. The method of Paragraph F, wherein the sugars are derived from cellulosic biomass
H. The method of Paragraph F or Paragraph G, wherein the fermentation is microbial fermentation
I. The method of any one of Paragraphs F-H, wherein the sugars comprise at least one of a simple sugar or an anhydrosugar.
J. The method of any one of Paragraphs A-I, wherein the fatty alcohols comprise at least 50 wt % of a mixture of 1-dodecanol and 1-tetradecanol.
K. The method of any one of Paragraphs A-J, wherein the fatty alcohols comprise about 1 wt % to about 5 wt % of one or more fatty alcohols containing a carbon-carbon double bond.
L. The method of any one of Paragraphs A-K, wherein the fatty alcohols comprise about 0.1 wt % to about 1 wt % diols.
M. The method of any one of Paragraphs A-L, wherein the feed further comprises at least two of a lipid, a biobased crude oil, or a petroleum fraction.
N. The method of any one of Paragraphs A-L, wherein the fatty alcohols are combined with a lipid, a biobased crude oil, a petroleum fraction, or combination of any two or more thereof, prior to hydrodeoxygenating the feed.
O. The method of any one of Paragraphs A-N, wherein the method further comprises hydroisomerizing the hydrocarbon product to produce a hydroisomerization product.
P. The method of Paragraph O, wherein the hydroisomerizing comprises contacting the hydrocarbon product with a hydroisomerization catalyst in a fixed-bed reactor.
Q. The method of Paragraph P, wherein the hydroisomerization catalyst comprises a bifunctional noble metal catalyst.
R. The method of Paragraph Q, wherein the bifunctional noble metal catalyst comprises platinum.
S. The method of Paragraph Q or Paragraph R, wherein the bifunctional noble metal catalyst comprises an acid-support.
T. The method of Paragraph S, wherein the acid-support comprises a zeolite.
U. The method of any one of Paragraphs Q-T, wherein the hydroisomerization catalyst comprises a base metal catalyst.
V. The method of Paragraph U, wherein the base metal catalyst comprises tungsten.
W. The method of Paragraph U or Paragraph V, wherein the base metal catalyst comprises an acid-support.
X. The method of Paragraph W, wherein the acid-support comprises a zeolite.
Y. The method of any one of Paragraphs O-X, wherein the hydroisomerizing comprises a temperature from about 580° F. to about 680° F.
Z. The method of any one of Paragraphs O-X, wherein the hydroisomerizing comprises a H₂ partial pressure of about 500 psig to about 2000 psig.
   Other embodiments are set forth in the following claims.

1. A method to produce hydrocarbon renewable fuels, the method comprising
hydrodeoxygenating a feed comprising fatty alcohols to produce a hydrocarbon product comprising C₁₀-C₁₂ n-paraffins and a heteroatom oxygen content less than 0.1 wt %.

2. The method of claim 1, wherein the hydrodeoxygenating comprises contacting the feed with a sulfided base metal catalyst at a temperature from about 500° F. to about 700° F. range and a hydrogen partial pressure from about 500 psig to about 4000 psig.

3. The method of claim 1, wherein the hydrocarbon renewable fuel comprises one or more of a renewable diesel, a renewable kerosene, and a renewable naphtha.

4.-5. (canceled)

6. The method of claim 1, wherein the feed comprises a product of fermentation of sugars.

7. The method of claim 6, wherein the sugars are derived from cellulosic biomass

8. The method of claim 6, wherein the fermentation is microbial fermentation

9. The method of claim 6, wherein the sugars comprise at least one of a simple sugar or an anhydrosugar.

10. The method of claim 1, wherein the fatty alcohols comprise at least 50 wt % of a mixture of 1-dodecanol and 1-tetradecanol.

11. The method of claim 1, wherein the fatty alcohols comprise about 1 wt % to about 5 wt % of one or more fatty alcohols containing a carbon-carbon double bond.

12. The method of claim 1, wherein the fatty alcohols comprise about 0.1 wt % to about 1 wt % diols.

13. The method of claim 1, wherein the feed further comprises at least two of a lipid, a biobased crude oil, or a petroleum fraction.

14. The method of claim 1, wherein the fatty alcohols are combined with a lipid, a biobased crude oil, a petroleum fraction, or combination of any two or more thereof, prior to hydrodeoxygenating the feed.

15. The method of claim 1, wherein the method further comprises hydroisomerizing the hydrocarbon product to produce a hydroisomerization product.

16. The method of claim 15, wherein the hydroisomerizing comprises contacting the hydrocarbon product with a hydroisomerization catalyst in a fixed-bed reactor.

17. The method of claim 16, wherein the hydroisomerization catalyst comprises a bifunctional noble metal catalyst.

18. The method of claim 17, wherein the bifunctional noble metal catalyst comprises platinum.

19.-20. (canceled)

21. The method of claim 17, wherein the hydroisomerization catalyst comprises a base metal catalyst.

22. The method of claim 21, wherein the base metal catalyst comprises tungsten.

23.-24. (canceled)

25. The method of claim 15, wherein the hydroisomerizing comprises a temperature from about 580° F. to about 680° F.

26. The method of claim 15, wherein the hydroisomerizing comprises a H₂ partial pressure of about 500 psig to about 2000 psig.
